(19) United States
(12) Patent Application Publication
(10) Pub. No.: US 2012/0201773 A1
(43) Pub. Date: Aug. 9, 2012

(54) UNENCUMBERING HAIR TREATMENT PRODUCT

(75) Inventors: Thorsten Knappe, Schenefeld (DE); Bernd Richters, Hamburg (DE)

(73) Assignee: Henkel AG & Co. KGaA, Dusseldorf (DE)

(21) Appl. No.: 13/451,672

(22) Filed: Apr. 20, 2012

Related U.S. Application Data
(63) Continuation of application No. PCT/EP2010/064992, filed on Oct. 7, 2010.

(30) Foreign Application Priority Data
Oct. 22, 2009 (DE) 10 2009 045 914.6

(51) Int. Cl. A61K 8/894; A61Q 5/00

(52) U.S. Cl. 424/70.12

(57) ABSTRACT

A hair treatment product includes in respect to the weight thereof, at least 70 wt % water, 0.1 to 1.5 wt % of at least one silicon-based water-in-oil emulsifier from the group of polyethylene-glycol modified silicon; and 1 to 10 wt % of cyclic siloxane. The product is significantly improved with respect to the care potential thereof and the effect thereof of fullness and volume.

### CROSS-REFERENCES TO RELATED APPLICATIONS

This application is a continuation of PCT/EP2010/064992, filed on Oct. 7, 2010, which claims priority under 35 U.S.C. §119 to DE 10 2009 045 914.6 filed on Oct. 22, 2009, both of which are hereby incorporated by reference.

### FIELD OF THE INVENTION

The present invention generally relates to agents for treatment of keratinic fibers.

### BACKGROUND OF THE INVENTION

Care products for keratinic fibers often have the disadvantage that they encumber the hair and thus reduce its body. This problem occurs in particular with products which are left on the hair (so-called leave-on products). In contrast with that, products that are rinsed out shortly after their use (so-called rinse-off products) often do not have adequate care potential. The object of the present invention was to provide hair treatment agents that would provide hair care for a long time, improve luster and feel without encumbering the hair style. Ideally this should be achievable with products that are rinsed out of the hair a relatively short period of time after being applied as well as those being left on the hair.

Another object of the present invention is to improve upon the instability of the compositions according to the prior art. The usual two-phase care products of the prior art have definite instabilities under a high mechanical stress. These stresses may include, for example, shaking prior to use or application from a spray head because high shearing forces occur in these processes. However, even at low temperatures, the usual compositions do not have adequate stability in particular when a high mechanical stress occurs additionally at low temperatures.

In an unforeseeable manner, it has now been discovered that the care product for hair containing an aqueous carrier can be improved significantly with regard to their care potential and their effect on the fullness and volume of the hair style as well as stability with respect to high mechanical stresses and low temperatures if they contain at least one cyclic silicone and at least one PEG dimethicone.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair treatment product, containing, based on its weight: at least 70 wt % water, 0.1 to 1.5 wt % of at least one silicone-based water-in-oil emulsifier from the group of polyethylene glycol-modified silicones, and 1 to 10 wt % cyclic siloxane.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is hair treatment agents containing, based on weight:
a) at least 70 wt % water,
b) 0.1 to 1.5 wt % of at least one silicone-based water-in-oil emulsifier from the group of polyethylene glycol-modified silicones, and
c) 1 to 10 wt % cyclic siloxane.

The agents according to the invention contain at least 70 wt % water. Agents preferred according to the invention are characterized in that they contain—based on their weight—75 to 97.5 wt %, preferably 77.5 to 95 wt %, more preferably 80 to 92.5 wt % and in particular 82.5 to 90 wt % water.

The agents according to the invention additionally contain at least one silicone-based water-in-oil emulsifier from the group of polyethylene glycol-modified silicones, the previous INCI designation of which was dimethicone copolyol, with the current INCI designations PEG-x dimethicone (where x=2-20, preferably 3-17, especially preferably 5-15). The commercial product Dow Corning ES-5612 formulation aid, for example, is especially preferred.

Preferred agents according to the invention contain silicone-based water-in-oil emulsifiers from the group of polyethylene glycol-modified silicones, preferably within narrow quantity ranges. The preferred agents according to the invention are those containing—based on their weight—0.15 to 0.25 wt %, preferably 0.2 to 1.0 wt %, more preferably 0.25 to 0.75 wt % and in particular 0.3 to 0.5 wt % of at least one silicone-based water-in-oil emulsifier from the group of polyethylene glycol-modified silicones.

Most especially preferred agents according to the invention contain—based on their weight—0.15 to 1.25 wt %, preferably 0.2 to 1.0 wt %, more preferably 0.25 to 0.75 wt % and in particular 0.3 to 0.5 wt % Dow Corning ES 5612 formulation aid.

Cyclic siloxanes, which are cyclic dimethicones known according to the INCI as cyclomethicones, can be used to advantage according to the invention. Cosmetic or dermatological preparations according to the invention, which contain at least one silicone of the formula (Si-4)

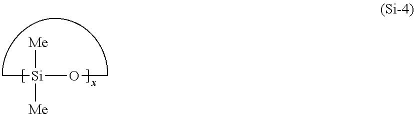

in which x stands for a number from 3 to 200, preferably from 3 to 10, more preferably from 3 to 7 and in particular 3, 4, 5 or 6 are preferred here. The cyclopentasiloxane (CAS 2,4,6,8,10-pentamethylcyclopentasiloxane) with x=5 is the most preferred.

The agents according to the invention contain at least 1 to 5 wt % of the cyclic siloxanes. Especially preferred agents according to the invention are characterized in that they contain—based on their weight—1.5 to 9.5 wt %, preferably 1.75 to 9 wt %, more preferably 2.0 to 8.5 wt %, even more preferably 2.5 to 8.0 wt %, even more preferably 3.0 to 7.5 wt % and in particular 3.5 to 7.5 wt % cyclic siloxanes.

In addition to the silicone-based water-in-oil emulsifier from the group of polyethylene glycol-modified silicones, the agents according to the invention contain 0.01 to 5 wt % of at least one silicon-free emulsifier.

Oil-in-water emulsifiers that are preferred according to the invention have an HLB value of at least 8, where the total oil-in-water emulsifier system preferably has a weight-average HLB value in the range of 11-17, preferably 13.5 to 15.5. These are the emulsifiers with which the skilled person is familiar in general such as those listed, for example, in Kirk Othmer, Encyclopedia of Chemical Technology, 3^{rd} edition, 1979, vol. 8, pages 913-916. For ethoxylated products the HLB value is calculated according to the equation HLB=(100−L):5, where L is the amount by weight of lipophilic groups, i.e., the fatty alkyl groups or fatty acyl groups in the ethylene oxide adducts, expressed in percent by weight.

In the selection of silicone-free oil-in-water emulsifiers that are suitable according to the invention, preferably nonionic oil-in-water emulsifiers, it is especially preferable to use a mixture of oil-in-water emulsifiers, preferably nonionic oil-in-water emulsifiers to be able to optimally adjust the stability of the compositions according to the invention. The individual emulsifier components supply a fraction to the total HLB value or average HLB value of the oil-in-water emulsifier mixture according to its weight proportion of the total weight of the oil-in-water emulsifiers. The weight-average HLB value of the oil-in-water emulsifier system according to the invention preferably amounts to 11-17, preferably 12-15 and especially preferably 13.5 to 15.5. To achieve such HLB values, oil-in-water emulsifiers from the HLB value ranges of 10-14, 14-16 and optionally 15-17 are combined. The oil-in-water emulsifier mixtures (or oil-in-water emulsifier systems) may of course also contain emulsifiers, preferably nonionic emulsifiers, with HLB values in the range of >7 10 and 17-20. Such emulsifier mixtures may also be preferred according to the invention. However, in another preferred embodiment, the compositions according to the invention may also contain just one single oil-in-water emulsifier with an HLB value in the range of 11-17, preferably 12-15 and especially preferably 13 14.

Preferred agents according to the invention are characterized in that the silicone-free oil-in-water emulsifiers are selected from ethoxylated C₈-C₂₄ alkanols with an average of 8-100 mol ethylene oxide per mol, ethoxylated C₈-C₂₄ carboxylic acids with an average of 8-100 mol ethylene oxide per mol, with an average of 20-100 mol ethylene oxide per mol ethoxylated glycerol mono- and/or diesters of linear saturated and unsaturated C₁₂-C₃₀ carboxylic acids which may be hydroxylated, in particular those of myristic acid, palmitic acid, stearic acid, 12-hydroxystearic acid or mixtures of their fatty acids with an average of 20-100 mol ethylene oxide per mol ethoxylated sorbitan monoesters of linear saturated and unsaturated C₁₂-C₃₀ carboxylic acids which may be hydroxylated, in particular those of myristic acid, palmitic acid, stearic acid, 12-hydroxystearic acid or mixtures of these fatty acids, silicone copolyols with ethylene oxide units or with ethylene oxide and propylene oxide units, alkyl mono- and oligoglycosides with 8 to 22 carbon atoms in the alkyl radical and their ethoxylated analogs, ethoxylated sterols, partial esters of polyglycerols with n=2 to 10 glycerol units and esterified with 1 to 4 saturated or unsaturated, linear or branched, optionally hydroxylated C₈-C₃₀ fatty acid esters, if they have an HLB value of more than 7, as well as mixtures of the aforementioned substances.

The ethoxylated C₈-C₂₄ alkanols have the formula R¹O—(CH₂CH₂O)ₙH wherein R¹ stands for a linear or branched alkyl and/or alkenyl radical with 8-24 carbon atoms, and n stands for the average number of ethylene oxide units per molecule, for numbers from 8-100, preferably 8-30 mol ethylene oxide onto 1 mol capryl alcohol, 2-ethylhexyl alcohol, capric alcohol, lauryl alcohol, isotridecyl alcohol, tridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol as well as their technical-grade mixtures. Adducts of 8-100 mol ethylene oxide onto technical-grade fatty alcohols with 12-18 carbon atoms, such as coconut, palm, palm kernel or tallow fatty alcohol are also suitable.

The ethoxylated C₈-C₂₄ carboxylic acids have the formula R¹O—(CH₂CH₂O)ₙH, wherein R¹O stands for a linear or branched, saturated or unsaturated acyl radical with 8-24 carbon atoms, and n stands for the average number of ethylene oxide units per molecule, for numbers from 8-100, preferably 10-30 mol ethylene oxide onto 1 mol caprylic acid, 2-ethylhexanoic acid, capric acid, lauric acid, isotridecanoic acid, myristic acid, cetyl acid, palmitoleic acid, stearic acid, isostearic acid, oleic acid, elaidic acid, petroselinic acid, arachic acid, gadoleic acid, behenic acid, erucaic acid and brassidic acid as well as their technical-grade mixtures. Adducts of 10-100 mol ethylene oxide onto technical-grade fatty acids with 12-18 carbon atoms such as coconut, palm, palm kernel or tallow fatty acid are also suitable. Especially preferred are PEG-50 monostearate, PEG-100 monostearate, PEG-50 monooleate, PEG-100 monooleate, PEG-50 monolaurate and PEG-100 monolaurate.

Especially preferred for use here are the C₁₂-C₁₈ alkanols or the C₁₂-C₁₈ carboxylic acids each with 8-30 units of ethylene oxide per molecule as well as mixtures of these substances, in particular laureth-8, laureth-10, laureth-12, laureth-20, trideceth-8, trideceth-9, trideceth-10, trideceth-12, trideceth-20, ceteth-10, ceteth-12, ceteth-20, ceteth-30, steareth-10, steareth-12, steareth-20, steareth-30, ceteareth-10, ceteareth-12, ceteareth-20, ceteareth-30, laureth-12 and beheneth-20.

Preferred glycerol mono- and/or diesters of linear saturated and unsaturated C₁₂-C₃₀ carboxylic acids ethoxylated with an average of 20-100 mol ethylene oxide per mol and optionally hydroxylated are selected from PEG-20 hydrogenated castor oil, PEG-40 hydrogenated castor oil and PEG-60 hydrogenated castor oil.

Preferred sorbitan monoesters of linear saturated and unsaturated C₁₂-C₃₀ carboxylic acids ethoxylated with an average of 20-100 mol ethylene oxide per mol and optionally hydroxylated are selected from polysorbate-20, polysorbate-40, polysorbate-60 and polysorbate-80.

In addition preferably C₉-C₂₂ alkyl mono- and oligoglycosides are used. C₈-C₂₂ alkyl mono- and oligoglycosides are known commercial surfactants and emulsifiers. They are prepared in particular by reacting glucose or oligosaccharides with primary alcohols having 8-22 carbon atoms. With respect to the glycoside radical, it is true that both monoglycosides in which a cyclic sugar radical is glycosidically bound to the fatty alcohol and oligomeric glycosides having a degree of oligomerization up to approx. 8 preferably 1-2 are suitable. The degree of oligomerization is a statistical mean based on a homolog distribution which is conventional for such technical-grade products. Products obtainable under the brand name Plantacare® contain a glucosidically bound C₈-C₁₆ alkyl group on an oligoglucoside radical, whose average degree of oligomerization is 1-2, in particular 1.2-1.4. Especially preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside as well as mixtures thereof.

Ethoxylated sterols, in particular ethoxylated soy sterols are suitable oil-in-water emulsifiers according to the invention. The degree of ethoxylation must be greater than 5, preferably at least 10 to have an HLB value greater than 7. Suitable commercial products include, for example, PEG-10 soy sterol, PEG-16 soy sterol and PEG-25 soy sterol.

In addition, partial esters of polyglycerols with 2 to 10 glycerol units and esterified with 1 to 4 saturated or unsaturated, linear or branched, optionally hydroxylated C₈-C₃₀ fatty acid esters are preferably used if they have an HLB value of more than 7. Especially preferred are diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol-monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Instead of or in addition to nonionic emulsifiers, ionic silicone-free emulsifiers may of course also be used. In particular cationic emulsifiers are preferred here (see below).

Regardless of the type of silicone-free emulsifier, agents according to the invention containing silicone-free emulsifiers within narrow quantity ranges are preferred. These preferred agents are characterized in that they contain—based on their weight—0.02 to 4.5 wt % preferably 0.03 to 4.0 wt %, more preferably 0.04 to 3.5 wt %, even more preferably 0.05 to 3.0 wt % and in particular 0.1 to 1.0 wt % of at least one silicone-free water-in-oil emulsifier.

As already mentioned, cationic compounds are preferred as the silicone-free emulsifiers. Agents according to the invention containing exclusively cationic compounds as the silicone-free emulsifiers are preferred here.

Through the use of quaternary surface-active compounds with an antimicrobial effect, the agent may be furnished with antimicrobial effect and/or its antimicrobial effect which is optionally already present due to other ingredients may be improved. Suitable quats here include, for example, benzalkonium chloride (N-alkyl-N,N-dimethylbenzylammonium chloride, CAS #8001-54-5), benzalkone B (m,p-dichlorobenzyldimethyl-C₁₂-alkylammonium chloride, CAS #58390-78-6), benzoxonium chloride (benzyldodecyl-bis-(2-hydroxyethyl)ammonium chloride), cetrimonium bromide (N-hexadecyl-N,N-trimethylammonium bromide, CAS #57-09-0), benzetonium chloride (N,N-dimethyl-N-[2-[2-[p(1,1,3,3-tetramethylbutyl)phenoxy]ethoxy]ethyl]benzylammonium chloride, CAS #121-54-0), dialkyldimethylammonium chlorides such as di-n-decyldimethylammonium chloride (CAS #7173-51-5-5), didecyldimethylammonium bromide (CAS #2390-68-3), dioctyldimethylammonium chloride, 1-cetylpyridinium chloride (CAS #123-03-5) and thiazoline iodide (CAS #15764-48-1) as well as mixtures thereof. Preferred quats include benzalkonium chlorides with C₈-C₁₈ alkyl radicals in particular C₁₂-C₁₄ alkylbenzyldimethylammonium chloride. An especially preferred quat is coconut pentaethoxymethylammonium methosulfate (INCI PEG-5 cocomonium methosulfate; Rewoquat® CPEM).

In addition, a quaternary imidazoline compound, i.e., a compound having a positively charged imidazoline ring is also present as the cationic surfactant. Formula I shown below gives the structure of these compounds

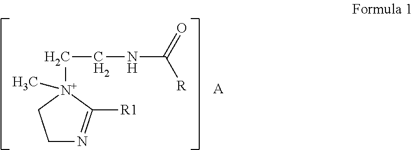

The radicals R and R1, independently of one another, each stand for a saturated or unsaturated, linear or branched hydrocarbon radical with a chain length of 8 to 30 carbon atoms. The preferred compounds of formula I each contain the same hydrocarbon radical for R and R1. The chain length of the radicals R and R1 is preferably 12 carbon atoms. Compounds with a chain length of at least 16 carbon atoms and most especially preferably with at least 20 carbon atoms are especially preferred. An extremely preferred compound of formula I has a chain length of 21 carbon atoms. A product of this chain length is available, for example, under the designation quatemium-91 or the commercial designations Crodazosoft® DBQ, containing, in addition to quaternium-91, also cetrimonium methosulfate and cetearyl alcohol as well as Crodazosoft® SCQ, which also contains PPG-3 benzyl ether myristate in addition to quatemium-91. In particular, examples according to the invention can be obtained, for example, under the INCI designations quaternium-27, quaternium-72, quatemium-83 and quaternium-91. The commercial products Crodazosoft® DBQ and Crodazosoft® SCQ and/or quaternium-91 are the most preferred.

The imidazolines of formula I are present in the compositions according to the invention in amounts of 0.01 to 20 wt %, preferably in amounts of 0.05 to 10 wt % and most especially preferably in amounts of 0.1 to 7.5 wt %. The best results are obtained with quantities of 0.1 to 5 wt %, each based on the total composition of the respective agent.

In addition the following cationic surfactants according to the formula (Tkat-2) may be used

RCO—X—N⁺R¹R²R³A⁻ (Tkat-2)

R here stands for a substituted or unsubstituted, branched or linear alkyl or alkenyl radical with 11 to 35 carbon atoms in the chain,
X stands for O or NR⁵,
R¹ stands for an alkylene group with 2 to 6 carbon atoms, which may be substituted or unsubstituted such that in the event of a substitution, the substitution with an OH group or an NH group is preferred,
R², R³, independently of one another, each represents an alkyl or hydroxyalkyl group with 1 to 6 carbon atoms in the chain, where the chain may be linear or branched.

R⁵ stands for hydrogen or a C₁ to C₆ linear or branched alkyl or alkenyl radical which may also be substituted by a hydroxyl group.

Within this structural class, the compounds of one of the following structures are preferably used:

CH₃(CH₂)₂₀CONH(CH₂)₃—N⁺(CH₃)₂—CH₂CH₃A (Tkat-3)

CH₃(CH₂)₂₀CONH(CH₂)₃—N⁺(CH₃)₂—CH₂(CHOH)CH₂OH A (Tkat-4)

CH₃(CH₂)₂₀COCHCHOHCH₂—N⁺(CH₃)₃A (Tkat-5)

CH₃(CH₂)₂₀CONH(CH₂)₃—N⁺(CH₃)₂—CH₂CH₂OH A (Tkat-6)

Examples of such commercial products include Schercoquat BAS, Lexquat AMG-BEO, Akypoquat 131 or Incroquat Behenyl HE.

Furthermore, ester quats according to the formula (Tkat1-2) may also be used

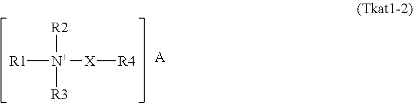

wherein the radicals R1, R2 and R3 are each independent of one another and may be the same or different. The radicals R1, R2 and R3 denote:
a branched or unbranched alkyl radical with 1 to 4 carbon atoms, which may contain at least one hydroxyl group or
a saturated or unsaturated branched or unbranched or a cyclic saturated or unsaturated alkyl radical with 6 to 30 carbon atoms, which may contain at least one hydroxyl group or
an aryl or alkaryl radical, for example, phenyl or benzyl or
the radical (X-R4) as defined below, with the provision that at most two of the radicals R1, R2 or R3 may stand for this radical.

The radical (X-R4) is present at least one to three times.

In this formula, X stands for:
1) (CH₂)ₙ, where n=1 to 20 preferably n=1 to 10 and especially preferably n=1-5, or
2) (CH₂—CHR5-O)ₙ, where n=1 to 200, preferably 1 to 100, especially preferably 1 to 50 and especially preferably 1 to 20, with R5 being present in the meaning of hydrogen, methyl or ethyl,
   and R4 stands for:
1) R6-O—CO, wherein R6 is a saturated or unsaturated, branched or unbranched or cyclic saturated or unsaturated alkyl radical with 6 to 30 carbon atoms, which may contain at least one hydroxyl group and which may optionally also be ethoxylated with 1 to 100 ethylene oxide units and/or 1 to 100 propylene oxide units, or
2) R7-CO, wherein R7 denotes a saturated or unsaturated, branched or unbranched or cyclic saturated or unsaturated alkyl radical with 6 to 30 carbon atoms, which may contain at least one hydroxyl group and which may also be ethoxylated with 1 to 100 ethylene oxide units and/or 1 to 100 propylene oxide units,
   and A stands for a physiologically tolerable organic or inorganic anion.

Such products are distributed, for example, under the brand names Rewoquat®, Stepantex®, Dehyquart® and Ainlocare®. The products Armocare® VGH-70, an N,N-bis(2-palmitoyloxyethyl)dimethylammonium chloride and Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such ester quats.

Additional compounds of the formula (Tkat1-2) which are especially preferred according to the invention fall under the formula (Tkat1-2.1), the cationic betaine esters.

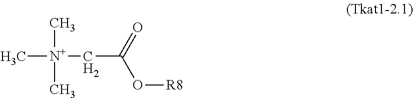

R8 corresponds in its meaning to R7.

As an additional ingredient, monoalkyl trimethylammonium salts with a chain length of the alkyl radical of 16 to 24 carbon atoms may also be present.

These compounds have the structure represented in the formula (Tkat1-1):

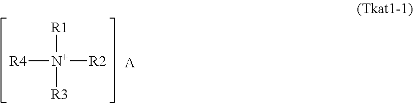

wherein R1, R2 and R3 each stand for a methyl group and R4 stands for a saturated, branched or unbranched alkyl radical with a chain length of 16 to 24 carbon atoms.

Examples of compounds of the formula (Tkat1-1) include cetyl trimethylammonium chloride, cetyl trimethylammonium bromide, cetyl trimethylammonium methosulfate, stearyl trimethylammonium chloride, behenyl trimethylammonium chloride, behenyl trimethylammonium bromide and behenyl trimethylammonium methosulfate.

In an especially preferred embodiment of the invention, the agents according to the invention additionally contain at least one amine and/or cationized amine in particular an amidoamine and/or a cationized amidoamine with the following structural formulas:

[R¹—NH—(CH₂)ₙ—NR²R³]A (Tkat7) and/or

[R¹—NH—(CH₂)ₙ—NR²R³R⁴]A (Tkat8)

wherein R1 may be an acyl or alkyl radical with 6 to 30 carbon atoms, which may be branched or unbranched, saturated or unsaturated, and wherein the acyl radical and/or the alkyl radical may contain at least one OH group, and
R2, R3 and R4, each independently of one another, denote hydrogen or an alkyl radical with 1 to 4 carbon atoms, which may be the same or different, saturated or unsaturated, and
A is an anion, and
n is an integer between 1 and 10.

A composition in which the amine and/or the quaternized amine according to general formulas (Tkat7) and/or (Tkat8) is preferably an amidoamine and/or a quaternized amidoamine wherein R1 denotes a branched or unbranched, saturated or unsaturated acyl radical with 6 to 30 carbon atoms, which may contain at least one OH group. A fatty acid radical of oils and waxes, in particular of natural oils and waxes is preferred here. Examples that might be considered for this include lanolin, beeswax or candellila wax.

Amidoamines and/or quaternized amidoamines in which R2, R3 and/or R4 in formulas (Tkat7) and/or (Tkat8) denote a radical according to the general formula CH₂CH₂OR5 wherein R5 may have the meaning of alkyl radicals with 1 to 4 carbon atoms, hydroxyethyl or hydrogen, are also preferred. The preferred size of n in the general formulas (Tkat7) and/or (Tkat8) is an integer between 2 and 5.

The alkyl radical with 1 to 4 carbon atoms of R2, R3 and R5 in the general formula (Tkat7) and/or (Tkat8) may contain at least one hydroxyl group.

The alkylamidoamines may be present as such and may be converted by protonation in suitably acidic solution into a quaternary compound in the composition. The cationic alkylamidoamine are preferred according to the invention.

As amidoamine to be used according to the invention, which may optionally be quaternized, examples that may be considered as amidoamines include: Witcamine 100 (Witco, INCI designation: cocamidopropyl dimethylamine), Incromine BB (Croda, INCI designation: behenamidopropyl dimethylamine), Mackine 401 (McIntyre, INCI designation: isostearylamidopropyl dimethylamine) and other Mackine products, Adogen S18V (Witco, INCO designation: stearylamidopropyl dimethylamine) and as permanently cationic amidoamines Rewoquat RTM 50 (Witco Surfactants GmbH, INCI designation: ricinoleamidopropyltrimonium methosulfate), Empigen CSC (Albright & Wilson, INCI designation: cocamidopropyltrimonium chloride), Swanol Lanoquat DES-50 (Nikko, INCI designation: quaternium-33), Rewoquat UTM 50 (Witco Surfactants GmbH, undecyleneamidopropyltrimonium methosulfate).

The anion A according to all the structural formulas given above for all the cationic compounds listed above is selected from the physiologically tolerable anions. Examples include the halide ions, fluoride, chloride, bromide, sulfate of the general formula RSO₃⁻ in which R has the meaning of saturated or unsaturated alkyl radicals with 1 to 4 carbon atoms, or anionic radicals of organic acids such as maleate, fumarate, oxalate, tartrate, citrate, lactate or acetate.

The cationic surfactants mentioned above may be used individually or in any combinations with one another, amounts between 0.01 and 20 wt %, preferably amounts of 0.01 to 10 wt % and most especially preferably amounts of 0.1 to 7.5 wt % being present. The best results are obtained with amounts of 0.1 to 5 wt %, each based on the total composition of the respective agent.

Most especially preferred agents according to the invention are characterized in that they contain as the silicone-free emulsifiers at least one cationic surfactant preferably at least one imidazolinium salt or at least one C₈₋₂₄ alkyl trimethylammonium salt, especially preferably C₁₀₋₂₀ alkyl trimethylammonium salt and even more preferably C₁₂₋₁₈ alkyl trimethylammonium salts and in particular cetyl trimethylammonium chloride or a mixture of at least one imidazolinium salts and a C₈₋₂₄ alkyl trimethylammonium salt.

Through the choice of the type and quantity of silicone-containing and silicone-free emulsifiers and their ratio to cyclodimethicone and optionally oil substances contained in the agent, the agents according to the invention may be formulated as a stable emulsion. However, it is also possible and preferable to supply the agents according to the invention in the form of an emulsion, which is at least partially unstable. This provides a two-phase product, which includes a lower aqueous phase and an upper oil phase. If the emulsion does not break completely, a two-phase product is formed, containing a clear aqueous bottom phase and a milky cloudy top phase of unbroken emulsion. Such products have a high optical differentiation and also have a great consumer acceptance. The consumer shakes the product prior to use, applies the emulsion, which is stable only briefly, to the hair, while the remainder of the emulsion in the product packaging separates again. Two-phase products according to the invention are especially preferred for these reasons; in other words, agents preferred according to the invention are not emulsions that are stable over a long period of time.

The care effects of the agents according to the invention may be further increased through the choice of a suitable pH value of the agents according to the invention. In particular acidic agents according to the invention have extraordinarily good care properties without encumbering the hair style. Agents preferred according to the invention are therefore characterized in that they have a pH of less than 5, preferably less than 4, more preferably 2.5 to 3.5 and in particular of 2.7 to 3.3.

As an additional optional component, the agents according to the invention may contain 0.01 to 10 wt % of at least one polymer from the group of cationic and/or amphoteric polymers.

The cationic polymers may be homopolymers or copolymers, wherein the quaternary nitrogen groups are present either in the polymer chain or preferably as a substituent on one or more of the monomers. The monomers containing ammonium groups cannot be copolymerized with non-cationic monomers. Suitable cationic monomers include unsaturated radically polymerizable compounds which have at least one cationic group, in particular ammonium-substituted vinyl monomers, for example, trialkyl methacryloxyalkylammonium, trialkyl acryloxyalkylammonium, dialkyl diallylammonium and quaternary vinyl ammonium monomers with cyclic, cationic groups containing nitrogen such as pyridinium, imidazolium or quaternary pyrrolidones, e.g., alkyl vinyl imidazolium, alkyl vinyl pyridinium or alkyl vinyl pyrrolidone salts. The alkyl groups of these monomers are preferably low alkyl groups, for example, C1 to C7 alkyl groups, especially preferably C1 to C3 alkyl groups.

The monomers containing ammonium groups may be copolymerized with noncationic monomers. Suitable comonomers include, for example, acrylamide, methacrylamide, alkyl and dialkylacrylamide, alkyl and dialkyl methacrylamide, alkyl acrylate, alkyl methacrylate, vinyl caprolactone, vinyl caprolactam, vinyl pyrrolidone, vinyl esters, such as vinyl acetate, vinyl alcohol, propylene glycol or ethylene glycol wherein the alkyl groups of these monomers are preferably C1 to C7 alkyl groups, especially preferably C1 to C3 alkyl groups.

Suitable polymers with quaternary amine groups include, for example, the polymers described under the designations polyquaternium in the CTFA Cosmetic Ingredient Dictionary, such as methyl vinyl imidazolium chloride-vinylpyrrolidone copolymer (polyquatemium-16) or quaternized vinylpyrrolidone-dimethyl aminoethyl methacrylate copolymer (polyquatemium-11).

Of the cationic polymers that may be present in the agents according to the invention, vinyl pyrrolidone-dimethyl aminoethyl methacrylate methosulfate copolymer, for example, which is distributed by the company Gaf Co., USA under the brand names Gafquat® 755 N and Gafquat® 734 and of which Gafquat® 734 is especially preferred are suitable. Additional cationic polymers include, for example, the copolymer of polyvinyl pyrrolidone and imidazolimine methochloride, which is distributed by the company BASF, Germany under the brand name Luviquat® HM 550, the terpolymer of dimethyl diallylammonium chloride, sodium acrylate and acrylamide, distributed by the company Calgon, USA under the brand name Merquat® Plus 3300 and the vinyl pyrrolidone-methacryl amidopropyl trimethylammonium chloride copolymer, distributed by the company ISP under the brand name Gafquat® HS 100.

Homopolymers of the general formula (P1),

{CH₂—[CR¹COO—(CH₂)ₘN⁽⁺⁾R²R³R⁴]}ₙX⁻

in which R¹=—H or —CH₃, R², R³ and R⁴, independently of one another, are selected from C₁₋₄ alkyl, alkenyl or hydroxyalkyl groups, m=1, 2, 3, or 4, n is a natural number and X⁻ is a physiologically tolerable organic or inorganic anion. Within the scope of these polymers, those that are preferred according to the invention are those for which at least one of the following conditions applies: R¹ stands for a methyl group, R², R³ and R⁴ stand for methyl groups, m has the value 2.

Examples of physiologically tolerable counterions X— include halide ions, sulfate ions, phosphate ions, methosulfate ions as well as organic ions such as lactate, citrate, tartrate and acetate ions. Halide ions are preferred, in particular chloride.

A particularly suitable homopolymer is poly(methacryloyloxyethyl trimethylammonium chloride), optionally crosslinked, with the INCI designation polyquaternium-37. Such products are available commercially under the brand names Rheocare® CTH (Cosmetic Rheologies) and Synthalen® CR (3V Sigma), for example.

The homopolymer is preferably used in the form of a nonaqueous polymer dispersion. Such polymer dispersions are commercially available under the brand names Salcare® SC 95 and Salcare® SC 96.

A copolymer that is preferred according to the invention is the crosslinked acrylamide methacryloyloxyethyl trimethyammonium chloride copolymer. Such copolymers are commercially available under the brand name Salcare® SC 92.

Suitable cationic polymers derived from natural polymers include cationic derivatives of polysaccharides, for example, cationic derivatives of cellulose, starch or guar. Also suitable are chitosan and chitosan derivatives. Cationic polysaccharides have the general formula (P-3) G-O—B—N⁽⁺⁾RₐR_{b}R_{c}X⁻ wherein

G is an anhydroglucose radical, for example, starch or cellulose anhydroglucose;
B is a divalent compound group, for example, alkylene, oxyalkylene, polyoxyalkylene or hydroxyalkylene;
Rₐ, R_{b} and R_{c} are, independently of one another, alkyl, aryl, alkylaryl, arylalkyl, alkoxyalkyl or alkoxyaryl, each with up to 18 carbon atoms, where the total number of carbon atoms in Rₐ, R_{b} and R_{c} is preferably max. 20;
X⁻ is a conventional counter anion and is preferably chloride.

A cationic cellulose is distributed by Amerchol under the brand name Polymer JR® 400 and has the INCI designation polyquaternium-10. Another cationic cellulose has the INCI designation polyquaternium-24 and is distributed by Amerchol under the brand name LM-200. Additional commercial products include the compounds Celquat® H 100, and Celquat® L 200. The aforementioned commercial products are preferred cationic celluloses. Additional preferred cationic celluloses are known under the INCI designations polyquaternium-67 and polyquaternium-72.

Suitable cationic guar derivatives are distributed under the commercial designation Jaguar® and have the INCI designation guar hydroxypropyl trimonium chloride. In addition especially suitable cationic guar derivatives are also available commercially from the Hercules under the brand name N-Hance. Additional cationic guar derivatives are distributed by Cognis under the brand name Cosmedia®. A preferred cationic guar derivative is the commercial product AquaCat® from the company Hercules. This raw material is a predissolved cationic guar derivative.

A suitable chitosan is distributed, for example, by the company Kyowa Oil & Fat Japan under the brand name Flonac®. A preferred chitosan salt is chitosonium pyrrolidone carboxylate which is distributed, for example, under the brand name Kytamer® PC by the company Amerchol, USA. Additional chitosan derivatives are available commercially under the brand names Hydagen® CMF, Hydagen® HCMF and Chitolam® NB/101.

Other preferred cationic polymers include, for example:
cationic alkyl polyglycosides,
cationized honey, for example, the commercial product Honeyquat® 50,
polymeric dimethyldiallylammonium salts and their copolymers with esters and amides of acrylic acid and methacrylic acid. The products commercially available under the brand names Merquat® 100 (poly(dimethyldiallylammonium chloride)) and Merquat® 550 (dimethyldiallylammonium chloride-acrylamide copolymer) are examples of such cationic polymers,
vinyl pyrrolidone/vinyl imidazolium methochloride copolymers, such as those available under the brand names Luviquat® FC 370, FC 550, FC 905 and HM 552,
quaternized polyvinyl alcohol,
as well as the polymers with quaternary nitrogen atoms in the main polymer chain known by the designations polyquatemium 2, polyquaternium 17, polyquaternium 18 and polyquaternium 27,
vinyl pyrrolidone/vinyl caprolactam-acrylate terpolymers such as those with acrylic acid esters and acrylic acid amides as the third monomer building block available commercially under the brand name Aquaflex® SF 40.

The copolymers of vinyl pyrrolidone such as those obtainable as the commercial products Copolymer 845 (manufacturer ISP), Gaffix® VC 713 (manufacturer ISP), Gafquat® ASCP 1011, Gafquat® HS 110, Luviquat® 8155 and Luviquat® MS 370 can also be used according to the invention.

In addition cationized protein hydrolysates may also be included with the cationic polymers, wherein the underlying protein hydrolysate may originate from animals, for example, from collagen, milk or keratin, from plants, for example, from wheat, corn, rice, potatoes, soy or almonds, from marine life forms, for example, from fish collagen or algae or biotechnologically produced protein hydrolysates. Typical examples of the cationic protein hydrolysates and derivatives according to the invention include the products covered by the INCI designations in the International Cosmetic Ingredient Dictionary and Handbook, (seventh edition 1997, The Cosmetic, Toiletry and Fragrance Association, 1101 17^{th} Street, N.W., Suite 300, Washington, D.C. 20036-4702) and available commercially.

The cationic polymers are preferably contained in the compositions according to the invention in amounts of 0.01 to 10 wt %, based on the total agent. Amounts of 0.05 to 5 wt % are especially preferred.

Regardless of whether or not the agents contain amphoteric polymers, additional preferred agents according to the invention are characterized in that they contain—based on their weight—0.05 to 7.5 wt %, preferably 0.1 to 5 wt %, especially preferably 0.2 to 3.5 wt % and in particular 0.25 to 2.5 wt % cationic polymer(s).

In summary, the agents according to the invention are preferably those which contain—based on their weight—0.05 to 7.5 wt %, preferably 0.1 to 5 wt %, especially preferably 0.2 to 3.5 wt % and in particular 0.25 to 2.5 wt % cationic polymer(s), where the preferred cationic polymer(s) is/are selected from
poly(methacryloyloxyethyl trimethylammonium chloride) (INCI: poly-quaterium-37) and/or
quaternized cellulose derivatives (INCI: polyquaternium 10) and/or
cationic alkyl polyglycosides and/or
cationized honey and/or
cationic guar derivatives and/or
polymeric dimethyl diallylammonium salts and their copolymers with esters and amides of acrylic acid and methacrylic acid and/or
copolymers of vinyl pyrrolidone with quaternized derivatives of dialkyl aminoalkyl acrylate and methacrylate and/or
vinyl pyrrolidone/vinyl imidazolium methochloride copolymers and/or
quaternized polyvinyl alcohol and/or
polyquaternium 2 and/or
polyquaternium-7 and/or
polyquaternium-16 and/or
polyquaternium 17 and/or
polyquaternium 18 and/or
polyquaternium 24 and/94
polyquaternium 27.

In addition amphoteric polymers may also be used as the polymers. The term amphoteric polymers is understood to include polymers having both free amino groups and free COOH or SO₃H groups in the molecule and capable of forming internal salts as well as zwitterionic polymers which contain quaternary ammonium groups and COO⁻ or SO₃⁻ groups in the molecule as well as those polymers which contain COOH or SO₃H groups and quaternary ammonium groups.

Amphoteric and/or cationic polymers preferred according to the invention include those polymers in which a cationic group is derived from at least one of the following monomers:

(i) monomers with quaternary ammonium groups of the general formula (Mono 1)

R¹—CH═CR²—CO—Z—(CₙH₂ₙ)—N⁽⁺⁾R²R³R⁴A⁽⁻⁾ (Mono 1)

in which R¹ and R², independently of one another, stand for hydrogen or a methyl group and R³, R⁴ and R⁵, independently of one another, stand for alkyl groups with 1 to 4 carbon atoms, Z is an NH group or an oxygen atom, N is an integer from 2 to 5 and A⁽⁻⁾ is the anion of an organic or inorganic acid,
(ii) Monomers with quaternary ammonium groups of the general formula (Mono2)

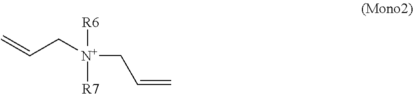

wherein R⁶ and R⁷, independently of one another, stand for a C₁ to C₄ alkyl group in particular for a methyl group and
A⁻ is the anion of an organic or inorganic,
(iii) monomeric carboxylic acids of the general formula (Mono3)

R⁸—CH═CR⁹—COOH (Mono3)

wherein R⁸ and R⁹, independently of one another, are hydrogen or methyl groups.

Especially preferred are those polymers in which the monomers of type (i) are used in which R³, R⁴ and R⁵ are methyl groups, Z is an NH group and A⁽⁻⁾ is a halide, methoxysulfate or ethoxysulfate ion; acrylamidopropyl trimethylammonium chloride is an especially preferred monomer (i). Acrylic acid is preferably used as monomer (ii) for the aforementioned polymers.

Especially preferred amphoteric polymers are copolymers from at least one monomer (Mono1) and/or (Mono2) with the monomer (Mono3) in particular copolymers of the monomers (Mono2) and (Mono3). Most especially preferred amphoteric polymers for use according to the invention are copolymer of diallyldimethylammonium chloride and acrylic acid. These copolymers are distributed under the INCI designation polyquaternium-22 with the brand name Merquat® 280 (Nalco), for example.

In addition, the amphoteric polymers according to the invention may also contain, in addition to a monomer (Mono1) or (Mono2) and a monomer (Mono3), a monomer (Mono4)

(iv) monomeric carboxylic acid amides of the general formula (Mono4)

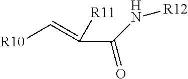

wherein R¹⁰ and R¹¹, independently of one another, are hydrogen or methyl groups and R¹² stands for a hydrogen atom or a C₁ to C₈ alkyl group.

Amphoteric polymers based on a copolymer (Mono4) that are most especially preferred for use according to the invention are the terpolymers of diallyl dimethylammonium chloride, acrylamide and acrylic acid. These copolymers are distributed under the INCI designation polyquaternium-39 with the brand name Merquat® Plus 3330 (Nalco), among others.

The amphoteric polymers may be used according to the invention in general either directly or in salt form, which is obtained by neutralization of the polymers, for example, with an alkali hydroxide.

The amphoteric polymers used in the agents according to the invention especially preferably contain monomers from the group of acrylamides and/or methacrylamides with alkylammonium groups. Acrylic acid and/or methacrylic acid and/or crotonic acid and/or 2-methylcrotonic acid have additionally proven successful as monomers with anionic groups additional contained in the polymer.

In summary, agents according to the invention in which the amphoteric polymer(s) is/are copolymers with at least one of the following monomers are preferred
trimethylammonium ethylacrylamide and/or
Trimethylammonium ethylmethacrylamide and/or
trimethylammonium propylacrylamide and/or
trimethylammonium propylmethacrylamide and/or
trimethylammonium ethylacrylamide and/or
trimethylammonium ethyl acrylate and/or
trimethylammonium ethyl methacrylate and/or
trimethylammonium ethyl acrylate and/or
ethyldimethylammonium ethylacrylamide and/or
ethyldimethylammonium ethylmethacrylamide and/or
ethyldimethylammonium propylacrylamide and/or
ethyldimethylammonium propylmethacrylamide and/or
ethyldimethylammonium ethylacrylamide and/or
ethyldimethylammonium ethyl acrylate and/or
ethyldimethylammonium ethylmethacrylate and/or
ethyldimethylammonium ethyl acrylate
   with at least one of the monomers
acrylic acid and/or
methacrylic acid and/or
crotonic acid and/or
2-methylcrotonic acid.

Especially preferred amphoteric polymers according to the invention are:
copolymers of trimethylammonium ethylacrylamide with acrylic acid
copolymers of trimethylammonium ethylacrylamide with methacrylic acid
copolymers of trimethylammonium ethylacrylamide with crotonic acid
copolymers of trimethylammonium ethylacrylamide with 2-methylcrotonic acid
copolymers of trimethylammonium ethylmethacrylamide with acrylic acid
copolymers of trimethylammonium ethylmethacrylamide with methacrylic acid
copolymers of trimethylammonium ethylmethacrylamide with crotonic acid
copolymers of trimethylammonium ethylmethacrylamide with 2-methylcrotonic acid
copolymers of trimethylammonium propylacrylamide with acrylic acid
copolymers of trimethylammonium propylacrylamide with methacrylic acid
copolymers of trimethylammonium propylacrylamide with crotonic acid
copolymers of trimethylammonium propylacrylamide with 2-methylcrotonic acid
copolymers of trimethylammonium propylmethacrylamide with acrylic acid
copolymers of trimethylammonium propylmethacrylamide with methacrylic acid
copolymers of trimethylammonium propylmethacrylamide with crotonic acid
copolymers of trimethylammonium propylmethacrylamide with 2-methylcrotonic acid
copolymers of trimethylammonium ethylacrylamide with acrylic acid
copolymers of trimethylammonium ethylacrylamide with methacrylic acid
copolymers of trimethylammonium ethylacrylamide with crotonic acid
copolymers of trimethylammonium ethylacrylamide with 2-methylcrotonic acid
copolymers of trimethylammonium ethyl acrylate with acrylic acid
copolymers of trimethylammonium ethyl acrylate with methacrylic acid
copolymers of trimethylammonium ethyl acrylate with crotonic acid
copolymers of trimethylammonium ethyl acrylate with 2-methylcrotonic acid
copolymers of trimethylammonium ethyl methacrylate with acrylic acid
copolymers of trimethylammonium ethyl methacrylate with methacrylic acid
copolymers of trimethylammonium ethyl methacrylate with crotonic acid
copolymers of trimethylammonium ethyl methacrylate with 2-methylcrotonic acid
copolymers of trimethylammonium ethyl acrylate with acrylic acid
copolymers of trimethylammonium ethyl acrylate with methacrylic acid
copolymers of trimethylammonium ethyl acrylate with crotonic acid
copolymers of trimethylammonium ethyl acrylate with 2-methylcrotonic acid
copolymers of ethyldimethylammonium ethylacrylamide with acrylic acid
copolymers of ethyldimethylammonium ethylacrylamide with methacrylic acid
copolymers of ethyldimethylammonium ethylacrylamide with crotonic acid
copolymers of ethyldimethylammonium ethylacrylamide with 2-methylcrotonic acid
copolymers of ethyldimethylammonium ethylmethacrylamide with acrylic acid
copolymers of ethyldimethylammonium ethylmethacrylamide with methacrylic acid
copolymers of ethyldimethylammonium ethylmethacrylamide with crotonic acid
copolymers of ethyldimethylammonium ethylmethacrylamide with 2-methylcrotonic acid
copolymers of ethyldimethylammonium propylacrylamide with acrylic acid
copolymers of ethyldimethylammonium propylacrylamide with methacrylic acid
copolymers of ethyldimethylammonium propylacrylamide with crotonic acid
copolymers of ethyldimethylammonium propylacrylamide with 2-methylcrotonic acid
copolymers of ethyldimethylammonium propylmethacrylamide with acrylic acid
copolymers of ethyldimethylammonium propylmethacrylamide with methacrylic acid
copolymers of ethyldimethylammonium propylmethacrylamide with crotonic acid
copolymers of ethyldimethylammonium propylmethacrylamide with 2-methylcrotonic acid
copolymers of ethyldimethylammonium ethylacrylamide with acrylic acid
copolymers of ethyldimethylammonium ethylacrylamide with methacrylic acid
copolymers of ethyldimethylammonium ethylacrylamide with crotonic acid
copolymers of ethyldimethylammonium ethylacrylamide with 2-methylcrotonic acid
copolymers of ethyldimethylammonium ethyl acrylate with acrylic acid
copolymers of ethyldimethylammonium ethyl acrylate with methacrylic acid
copolymers of ethyldimethylammonium ethyl acrylate with crotonic acid
copolymers of ethyldimethylammonium ethyl acrylate with 2-methylcrotonic acid
copolymers of ethyldimethylammonium ethyl methacrylate with acrylic acid
copolymers of ethyldimethylammonium ethyl methacrylate with methacrylic acid
copolymers of ethyldimethylammonium ethyl methacrylate with crotonic acid
copolymers of ethyldimethylammonium ethyl methacrylate with 2-methylcrotonic acid
copolymers of ethyldimethylammonium ethyl acrylate with acrylic acid
copolymers of ethyldimethylammonium ethyl acrylate with methacrylic acid
copolymers of ethyldimethylammonium ethyl acrylate with crotonic acid
copolymers of ethyldimethylammonium ethyl acrylate with 2-methylcrotonic acid

Preferably the amphoteric polymer(s) is/are used within narrow quantity ranges. Thus, for example, agents which contain—based on their weight—0.05 to 7.5 wt %, preferably 0.1 to 5 wt %, especially preferably 0.2 to 3.5 wt % and in particular 0.25 to 2.5 wt % amphoteric polymer(s) are preferred.

The anionic polymers are anionic polymers which have carboxylate groups and/or sulfonate groups. Examples of anionic monomers of which such polymers may consist include acrylic acid, methacrylic acid, crotonic acid, maleic anhydride and 2-acrylamido-2-methylpropane sulfonic acid. The acidic groups may be present entirely or in part as the sodium, potassium, ammonium, mono- or triethanolammonium salts. Preferred monomers are 2 acrylamido-2-methylpropane sulfonic acid and acrylic acid.

Anionic polymers containing 2-acrylamido-2-methylpropane sulfonic acid as the sole or comonomer have proven to be especially effective, where the sulfonic acid group may be present entirely or partially as the sodium, potassium, ammonium, mono- or triethanolammonium salt.

The homopolymer of 2-acrylamido-2-methylpropanesulfonic acid which is commercially available under the brand name Rheothik® 11-80, for example, is especially preferred.

Within this embodiment, it may be preferable to use copolymers of at least one anionic monomer and at least nonionic monomer. With respect to the anionic monomers, reference is made to the substances listed above. Preferred nonionic monomers include acrylamide, methacrylamide, acrylic acid esters, methacrylic acid esters, vinyl pyrrolidone, vinyl ethers and vinyl esters.

Preferred anionic copolymers are acrylic acid acrylamide copolymers as well as in particular polyacrylamide copolymers with monomers that contain sulfonic acid groups. Such a polymer is contained in the commercial product Sepigel® 305 from the company SEPPIC.

The sodium acryloyl dimethyl taurate copolymers distributed under the brand name Simulgel® 600 as a compound with isohexadecane and polysorbate 80 have also proven to be especially effective according to the invention.

Likewise preferred anionic homopolymers are crosslinked and uncrosslinked polyacrylic acid. Allyl ethers of pentaerythritol, sucrose and propylene may be preferred crosslinking agents. Such compounds are available commercially under the brand name Carbopol®, for example.

Copolymers of maleic anhydride and methyl vinyl ether, in particular those with crosslinking, are also color maintaining polymers. A maleic acid methyl vinyl ether copolymer crosslinked with 1,9-decadienes is available commercially under the brand name Stabileze® QM.

The anionic polymers are preferably used in the agents according to the invention in amounts of 0.05 to 10 wt %, based on the total agent. Amounts of 0.1 to 5 wt % are especially preferred.

A polyurethane that is most especially preferred according to the invention is commercially available under the brand name Luviset® PUR (BASF).

The agents according to the invention may contain nonionic polymers in another embodiment.

Suitable nonionic polymers include, for example:
Vinyl pyrrolidone/vinyl ester copolymers such as those distributed under the brand name Luviskol® (BASF). Luviskol® VA 64 and Luviskol® VA 73, both being vinylpyrrolidone vinyl acetate copolymers, are also preferred nonionic polymers.
Cellulose ethers such as hydroxypropyl cellulose, hydroxyethyl cellulose and methylhydroxypropyl cellulose such as those distributed, for example, under the brand names Culminal® and Benecel® (AQUALON) and Natrosol® products (Hercules);
Starch and derivatives thereof in particular starch ethers, for example, Structure® XL (National Starch), a multifunctional salt-tolerant starch;
Shellac
Polyvinyl pyrrolidones such as those distributed under the brand name Luviskol® (BASF), for example.

The nonionic polymers are preferably present in the compositions according to the invention in amounts of 0.05 to 10 wt %, based on the total agent. Amounts of 0.1 to 5 wt % are especially preferred.

It is also possible according to the invention for the preparations used to contain several polymers, in particular two different polymers of the same charge and/or an anionic polymer and an amphoteric polymer and/or a nonionic polymer.

In another preferred embodiment of the invention, the effect of the active ingredient according to the invention may be further optimized by fat substances. Fat substances are understood to be fatty acids, fatty alcohols, natural and synthetic waxes which may be present in both solid form and liquid form in aqueous dispersion as well as natural and synthetic cosmetic oil components.

The fatty acids used may be linear and/or branched, saturated and/or unsaturated fatty acids with 6 to 30 carbon atoms. Fatty acids with 10 to 22 carbon atoms are preferred. These include, for example, the isostearic acids such as the commercial product Emersol® 871 and Emersol® 875 and isopalmitic acids such as the commercial product Edenor® IP 95 as well as all other fatty acids distributed under the brand names Edenor® (Cognis). Other typical examples of such fatty acids include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucaic acid as well as their technical-grade mixtures which are obtained, for example, in pressure cleavage of natural fats and oils, in the oxidation of aldehydes from Roelen's oxosynthesis or dimerization of unsaturated fatty acids. The fatty acid cuts that are obtained from coconut oil or palm oil are especially preferred. The use of stearic acid is usually preferred in particular.

The quantity used is 0.1-15 wt %, based on the total agent. In a preferred embodiment, the quantity is 0.5-10 wt %, but quantities of 1-5 wt % are most especially advantageous.

Saturated, mono- or polyunsaturated, branched or unbranched fatty alcohols with C₆-C₃₀, preferably C₁₀-C₂₂ and most especially C₁₂-C₂₂ carbon atoms may be used as the fatty alcohols. In the sense of the invention, decanol, octanol, octenol, dodecenol, decenol, octadienol, dodecadienol, decadienol, oleyl alcohol, erucaic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol as well as their Guerbet alcohols may be used in the sense of this invention, but this list is intended only as an example and should not restrict the invention in any way. However, the fatty alcohols are derived from preferably naturally fatty acids, and it may usually be assumed that they are produced from the esters of the fatty acids by reduction. Likewise fatty alcohol cuts which are created by reduction of naturally occurring triglycerides such as beef tallow, palm oil, peanut oil, canola oil, cottonseed oil, soy oil, sunflower oil and linseed oil or the fatty acid esters formed from their transesterification products with the corresponding alcohols may also be used according to the invention, and thus they constitute a mixture of different fatty alcohols. Such substances are available commercially, for example, under the brand names Stenol®, e.g., Stenol® 1618 or Lanette®, e.g., Lanette® O or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Lanolin alcohols such as those available commercially under the brand names Corona®, White Swan®, Coronet or Fluilan® may of course also be used according to the invention. The fatty alcohols are used in amounts of 0.1-20 wt %, based on the total preparation, preferably in amounts of 0.1-10 wt %.

According to the invention, solid paraffins or isoparaffins, carnauba waxes, beeswax, candelilla waxes, ozokerites, ceresins, spermaceti, sunflower wax, fruit waxes such as apple wax or citrus wax, microwaxes of PE or PP may also be used as natural or synthetic waxes according to the invention. Such waxes are available commercially, for example, from the company Kahl & Co. Trittau.

Natural and synthetic cosmetic oil substances which can increase the effect of the active ingredient according to the invention include, for example:
Vegetable oils. Examples of such oils include sunflower oil, olive oil, soy oil, canola oil, almond oil, jojoba oil, orange oil, wheat germ oil, peach pit oil and the liquid fractions of coconut oil. However, other triglycerides are also suitable such as the liquid fraction of beef tallow as well as synthetic triglyceride oils;
Liquid paraffin oils, isoparaffin oils and synthetic hydrocarbons as well as di-n-alkyl ethers with a total of between 12 and 36 carbon atoms, in particular 12 to 24 carbon atoms such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert-butyl-n-octyl ether, isopentyl-n-octyl ether and 2-methylpentyl-n-octyl ether. The compounds 1,3-di-(2-ethylhexyl)cyclohexane (Cetiol® S) and di-n-octyl ether (Cetiol® OE) which are available commercially may be preferred;
Ester oils. Ester oils are understood to refer to the esters of C₆-C₃₀ fatty acids with C₂-C₃₀ fatty alcohols. The monoesters of fatty acids without alcohols with 2 to 24 carbon atoms are preferred. Examples of fatty acid components that may be used in the esters include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucaic acid as well as their technical-grade mixtures. Examples of the fatty alcohol fractions in the ester oils include isopropyl alcohol, capric alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol as well as their technical-grade mixtures. Especially preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V);
Dicarboxylic acid esters such as di-n-butyl adipate, di-(2-ethylhexyl) adipate, di-(2-ethylhexyl) succinate and diisotridecyl acelaate as well as diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate;
Symmetrical, asymmetrical or cyclic esters of carbonate acid with fatty alcohols, for example, glycerol carbonate or dicaprylyl carbonate (Cetiol® CC);
Mono-, di- and trifatty acid esters of saturated and/or unsaturated, linear and/or branched fatty acids with glycerol, for example, Monomuls® 90-018, Monomuls® 90-L12 or Cutina® MD.

The use quantity is 0.1-50 wt %, based on the total agent preferably 0.1-20 wt % and especially preferably 0.1-15 wt %, based on the total agent.

The total amount of oil and fat components in the agents according to the invention is usually 6-45 wt %, based on the total agent. Amounts of 10-35 wt % are preferred according to the invention.

In addition, it has been found that the effect of the active ingredient according to the invention can be increased when it is combined with hydroxycarboxylic acid esters. Preferred hydroxycarboxylic acid esters are full esters of glycolic acid, lactic acid, malic acid, tartaric acid or citric acid. Other fundamentally suitable hydroxycarboxylic acids esters include the esters of β-hydroxypropionic acid, tartronic acid, D-gluconic acid, saccharic acid, mucic acid or glucuronic acid. Suitable alcohol components of these esters include primary, linear or branched aliphatic alcohols with 8-22 carbon atoms, e.g., fatty alcohols or synthetic fatty alcohols. The esters of C₁₂-C₁₅ fatty alcohols are especially preferred. Esters of this type are commercially available, for example, under the brand name Cosmacol® from EniChem, Augusta Industriale. The quantity used of the hydroxycarboxylic acid esters is 0.1-15 wt %, based on the agent, preferably 0.1-10 wt % and most especially preferably 0.1-5 wt %.

As additional ingredients, the agents according to the invention may especially preferably contain one or more amino acids. Especially preferred amino acids for use according to the invention are obtained from the group glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cys), L-carnitine, L-citruline, L-threonine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, such that both the individual amino acids and also mixtures may be used.

Preferred agents according to the invention contain one or more amino acids in narrow quantity ranges. The preferred hair treatment agents according to the invention are characterized in that they contain as the care substance—based on their weight—0.01 to 5 wt % preferably 0.02 to 2.5 wt %, especially preferably 0.05 to 1.5 wt %, more preferably 0.075 to 1 wt % and in particular 0.1 to 0.25 wt % amino acid(s), preferably from the group of glycine and/or alanine and/or valine and/or lysine and/or leucine and/or threonine

Another preferred group of ingredients of the agents according to the invention are the vitamins, provitamins or vitamin precursors which are described below:

The group of substances referred to as vitamin A includes retinol (vitamin A1) and 3,4-didehydroretinol (vitamin A2). The β-carotene is the provitamin of retinol. For example, vitamin A acid and its esters, vitamin A aldehyde and vitamin A alcohol as well as its esters and the palmitate and acetate may be considered as the vitamin A component according to the invention. The agents according to the invention contain the vitamin A component preferably in amounts of 0.05-1 wt %, based on the total preparation.

The vitamin B group or the vitamin B complex includes, among others:
Vitamin B1 (thiamine);
Vitamin B2 (riboflavin);
Vitamin B3. This designation often includes the compounds nicotinic acid and nicotinamide (niacinamide). According to the invention, nicotinamide is preferred and is present in the agents according to the invention preferably in amounts of 0.05 to 1 wt %, based on the total agent;
Vitamin B5 (pantothenic acid, panthenol and pantolactone). Of this group, panthenol and/or pantolactone are preferred for use here. Derivatives of panthenol that may be used according to the invention include in particular the esters and ethers of panthenol as well as cationically derivatized panthenols. Individual representatives include, for example, panthenol triacetate, panthenol monoethyl ether and its monoacetate as well as the cationic panthenol derivatives. The afore mentioned compounds of the vitamin B5 type are preferably present in the agents according to the invention in amounts of 0.05-10 wt %, based on the total agent. Amounts of 0.1-5 wt % are especially preferred;
Vitamin B6 (pyridoxine and pyridoxamine and pyridoxal).

Vitamin C (ascorbic acid). Vitamin C is preferably used in the agents according to the invention in amounts of 0.1 to 3 wt %, based on the total agent. Use in the form of palmitic acid ester, the glucosides or phosphates may be preferred. Use in combination with tocopherols may also be preferred.

Vitamin E (tocopherol in particular α-tocopherol). Tocopherol and its derivatives including in particular the esters such as the acetate, the nicotinate, the phosphate and the succinate, are preferably present in the agents according to the invention in amounts of 0.05-1 wt %, based on the total agent.

Vitamin F. The term “vitamin F” is usually understood to refer to essential fatty acids, in particular linoleic acid, linolenic acid and arachidonic acid.

Vitamin H. The compound (3aS,4S,6aR)-2-oxohexahydrothienol[3,4-d]-imidazole-4-valeric acid is also referred to as vitamin H but in the meantime the trivial name biotin has become popular for this substance. Biotin is preferably contained in the agents according to the invention in amounts of 0.0001 to 1.0 wt %, in particular in amounts of 0.001 to 0.01 wt %.

In summary, hair treatment agents according to the invention, which additionally contain as a care substance—based on weight—in the amount of 0.1 to 5 wt %, preferably 0.2 to 4 wt %, especially preferably 0.25 to 3.5 wt %, more preferably 0.5 to 3 wt % and in particular 0.5 to 2.5 wt % vitamins and/or provitamins and/or vitamin precursors which are preferably assigned to the groups A, B, C, E, F and H are preferred according to the invention, wherein preferred agents also contain panthenol ((±)-2,4-dihydroxy-N-(3-hydroxypropyl)-3,3-dimethylbutyramide, provitamin B5) and/or pantothenic acid (vitamin B3, vitamin B5) and/or niacin, niacinamide and/or nicotinamide (vitamin B3) and/or L-ascorbic acid (vitamin C) and/or thiamine (vitamin B1) and/or riboflavin (vitamin B2, vitamin G) and/or biotin (vitamin B7, vitamin H) and/or folic acid (vitamin B9, vitamin Bc or vitamin M) and/or vitamin B6 and/or vitamin B12.

It has been found that the use of certain quinones potentiates an antidandruff effect and a hair loss-preventing effect and has advantages with respect to combability and luster. Therefore, the agents according to the invention may be understood to be additional ingredients containing 0.0001 to 5 wt % of at least one bioquinone and/or plastoquinone. The ubiquinones which are preferred according to the invention have the following formula:

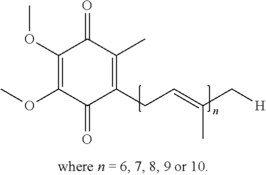

Coenzyme Q10 is the most preferred here.

To improve the elasticity and hair-setting effect of the internal structure of hair treated with the agents according to the invention, the agents according to the invention may contain purine and/or purine derivatives. In particular the combination of purine and/or purine derivatives with ubiquinones and/or plastoquinones results in the hair that has been treated with the corresponding agents having higher measured values in differential thermal analysis and improved wet combability and dry combability, among other things.

Preferred compositions according to the invention contain purine and/or purine derivatives in narrow quantity ranges. Cosmetic agents preferred according to the invention here are characterized in that they contain—based on their weight—0.001 to 2.5 wt %, preferably 0.0025 to 1 wt %, especially preferably 0.005 to 0.5 wt % and in particular 0.01 to 0.1 wt % purine(s) and/or purine derivative(s). Cosmetic agents preferred according to the invention are characterized in that they contain purine, adenine, guanine, uric acid, hypoxanthine, 6-purinethiol, 6 thioguanine, xanthine, caffeine, theobromine or theophylline. Caffeine is the most preferred in hair cosmetic preparations.

It is additionally advantageous to use purine and/or purine derivatives and bioquinones in a certain ratio to one another. Agents according to the invention in which the weight ratio of ingredients a) and b) is 10:1 to 1:100, preferably 5:1 to 1:50, especially preferably 2:1 to 1:20 and in particular 1:1 to 1:10 are preferred.

As already mentioned, caffeine is an especially preferred purine derivative and coenzyme Q10 is an especially preferred bioquinone. Especially preferred agents according to the invention are therefore characterized in that they contain—based on their weight—0.001 to 2.5 wt %, preferably 0.0025 to 1 wt %, especially preferably 0.005 to 0.5 wt % and in particular 0.001 to 0.1 wt % caffeine and 0.0002 to 4 wt %, preferably 0.0005 to 3 wt %, especially preferably 0.001 to 2 wt %, more preferably 0.0015 to 1 and in particular 0.002 to 0.5 wt % coenzyme Q10.

As an additional component, the agents according to the invention may contain at least one carbohydrate from the group of monosaccharides, disaccharides and/or oligosaccharides. Preferred hair treatment agents according to the invention are characterized in that they contain as the care substance—based on their weight—0.01 to 5 wt %, preferably 0.05 to 4.5 wt %, especially preferably 0.1 to 4 wt %, more preferably 0.5 to 3.5 wt % and in particular 0.75 to 2.5 wt % carbohydrate(s), selected from monosaccharides, disaccharides and/or oligosaccharides, but preferred carbohydrates are selected from
monosaccharides, in particular
   D-ribose and/or
   D-xylose and/or
   L-arabinose and/or
   D-glucose and/or
   D-mannose and/or
   D-galactose and/or
   D-fructose and/or
   sorbose and/or
   L-fucose and/or
   L-rhamnose
disaccharides, in particular
   sucrose and/or
   maltose and/or
   lactose and/or
   trehalose and/or
   cellobiose and/or
   gentiobiose and/or
   isomaltose.

Especially preferred agents according to the invention contain, based on their weight
0.005 to 0.015 wt % caffeine and 0.75 to 1.5 wt % glucose monohydrate,
0.005 to 0.015 wt % caffeine and 0.75 to 1.5 wt % sucrose,
0.005 to 0.015 wt % caffeine and 0.75 to 1.5 wt % fructose.

As already mentioned, preferred agents according to the invention contain (an) amino acid(s).

Especially preferred amino acids for use according to the invention are obtained from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrullin, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyro-glutamic acid), L-phosphoserine, creatine, 3-methyl-L-histidine, L-ornithine, wherein both the individual amino acids and also mixtures may be used.

Preferred agents according to the invention contain one or more amino acids in narrow quantity ranges. Preferred cosmetic agents according to the invention here are characterized in that they additional contain—based on their weight—0.05 to 5 wt %, preferably 0.1 to 2.5 wt %, especially preferably 0.15 to 1 wt % and in particular 0.2 to 0.5 wt % amino acid(s), preferably (an) amino acid(s) from the group of glycine and/or alanain and/or valine and/or lysine and/or leucine and/or threonine.

Especially preferred agents according to the invention contain, based on their weight
0.005 to 0.15 wt % caffeine and 0.75 to 1.5 wt % glucose monohydrate and 0.1 to 0.25 wt % glycine,
0.005 to 0.15 wt % caffeine and 0.75 to 1.5 wt % sucrose and 0.1 to 0.25 wt % glycine,
0.005 to 0.15 wt % caffeine and 0.75 to 1.5 wt % fructose and 0.1 to 0.25 wt % glycine,
0.005 to 0.15 wt % caffeine and 0.75 to 1.5 wt % glucose monohydrate and 0.1 to 0.25 wt % alanine,
0.005 to 0.15 wt % caffeine and 0.75 to 1.5 wt % sucrose and 0.1 to 0.25 wt % alanine,
0.005 to 0.15 wt % caffeine and 0.75 to 1.5 wt % fructose and 0.1 to 0.25 wt % alanine,
0.005 to 0.15 wt % caffeine and 0.75 to 1.5 wt % glucose monohydrate and 0.1 to 0.25 wt % valine,
0.005 to 0.15 wt % caffeine and 0.75 to 1.5 wt % sucrose and 0.1 to 0.25 wt % valine,
0.005 to 0.15 wt % caffeine and 0.75 to 1.5 wt % fructose and 0.1 to 0.25 wt % valine.

Taurine is another preferred care substance that may be used and has activating properties. Hair treatment agents preferred according to the invention contain as the care substance—based on their weight—0.01 to 15 wt %, preferably 0.025 to 12.5 wt %, especially preferably 0.05 to 10 wt %, more preferably 0.1 to 7.5 wt % and in particular 0.5 to 5 wt % taurine (2-aminoethane sulfonic acid).

The additional use of bisabolol and/or bisabololoxides in the agents according to the invention is also preferred. Hair treatment agents according to the invention, which additionally contain 0.001 to 5 wt %, preferably 0.01 to 4 wt %, especially preferably 0.02 to 2.5 wt % and in particular 0.1 to 1.5 wt % bisabolol and/or oxides of bisabolol, preferably (−)-α-bisabolol, are preferred here:

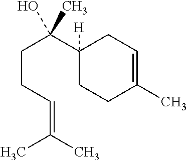

In addition, in a preferred embodiment of the invention, an agent according to the invention may also contain UV filters (I). The UV filters to be used according to invention are not subject to any general restrictions with regard to their structure and their physical properties. Instead all the UV filters that may be used in the cosmetic field, whose absorption maximum is in the UVA (315 400 nm) range, in the UVB (280-315 nm) range or in the UVC (<280 nm) range are suitable. UV filters with an absorption maximum in the UVB range in particular in the range of approx. 280 nm to approx. 300 nm are especially preferred.

The UV filters to be used according to the invention may be selected for example from substituted benzophenones, p-aminobenzoic acid esters, diphenylacrylic acid esters, cinnamic acid esters, salicylic acid esters, benzimidazoles and o-aminobenzoic acid esters.

Examples of UV filters that can be used according to the invention include 4-aminobenzoic acid, N,N,N-trimethyl-4-(2-oxoborn-3-ylidenemethyl)aniline methyl sulfate, 3,3,5-trimethylcyclohexyl salicylate (homosalate), 2-hydroxy-4-methoxybenzophenone (benzophenone-3; Uvinul® M 40, Uvasorb® MET, Neo Heliopan® BB, Eusolex® 4360), 2-phenylbenzimidazole5-sulfonic acid and its potassium, sodium and triethanolamine salts (phenylbenzimidazole sulfonic acid; Parsol® HS; Neo Heliopan® Hydro), 3,3′-(1,4-phenylenedimethylene)-bis(7,7-dimethyl-2-oxobicyclo[2.2.1]hept-1-ylmethanesulfonic acid) and salts thereof, 1-(4-tert-butylphenyl)-3-(4-methoxyphenyl)propane-1,3-dione (butyl methoxydibenzoylmethane; Parsol® 1789, Eusolex® 9020), α-(2-oxoborn-3-ylidene)toluene-4-sulfonic acid and its salts, ethoxylated 4-aminobenzoic acid ethyl ester (PEG-25 PABA; Uvinul® P 25), 4-dimethylaminobenzoic acid 2-ethylhexyl ester (octyl dimethyl PABA; Uvasorb® DMO, Escalol® 507, Eusolex® 6007), salicylic acid 2-ethylhexyl ester (octyl salicylate; Escalol® 587, Neo Heliopan® OS, Uvinul® 018), 4-methoxycinnamic acid isopentyl ester (isoamyl p-methoxycinnamate; Neo Heliopan® E 1000), 4-methoxycinnamic acid 2-ethylhexyl ester (octyl methoxycinnamate; Parsol® MCX, Escalol® 557, Neo Heliopan® AV), 2-hydroxy-4-methoxybenzophenone-5-sulfonic acid and its sodium salt (benzophenone-4; Uvinul® MS 40; Uvasorb® S 5), 3-(4′-methylbenzylidene)-D,L-camphor (4-methylbenzylidene camphor; Parsol® 5000, Eusolex® 6300), 3-benzylidene camphor (3-benzylidene camphor), 4-isopropylbenzyl salicylate, 2,4,6-trianilino-(p-carbo2′-ethylhexyl-1′-oxi)-1,3,5-triazine, 3-imidazol-4-ylacrylic acid and its ethyl ester, polymer of N-{(2 and 4)-[2-oxoborn-3-ylidenemethyl]benzyl}acrylamide, 2,4-dihydroxybenzophenone (benzophenone-1; Uvasorb® 20 H, Uvinul® 400), 1,1′-diphenylacrylonitrilic acid 2-ethylhexyl ester (octocrylene; Eusolex® OCR, Neo Heliopan® type 303, Uvinul® N 539 SG), o-aminobenzoic acid menthyl ester (menthyl anthranilate; Neo Heliopan® MA), 2,2′,4,4′-tetrahydroxybenzophenone (benzophenone-2; Uvinul® D-50), 2,2′-dihydroxy-4,4′-dimethoxybenzophenone (benzophenone-6), 2,2′-dihydroxy-4,4′-dimethoxybenzophenone-5-sodium sulfonate and 2-cyano-3,3-diphenylacrylic acid 2′-ethylhexyl ester. Preferred are 4-aminobenzoic acid, N,N,N-trimethyl-4-(2-oxobom-3-ylidenemethyl)aniline methyl sulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethylene)-bis(7,7-dimethyl-2-oxobicyclo[2.2.1]hept-1-ylmethanesulfonic acid) and its salts, 1-(4-tert-butylphenyl)-3-(4-methoxyphenyl)propane-1,3dione, α-(2-oxobom-3-ylidene)toluene-4-sulfonic acid and its salts, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and its sodium salt, 3-(4′-methylbenzylidene)-D,L-camphor, 3-benzylidene camphor, 4-isopropylbenzyl salicylate, 2,4,6-trianilino-(p-carbo-T-ethylhexyl-1′-oxi)-1,3,5-triazine, 3-imidazol-4-ylacrylic acid and its ethyl ester, polymer of N-{(2 and 4)-[2-oxoborn-3-ylidenemethyl]benzyl}acrylamide. Most especially preferred according to the invention are 2-hydroxy-4-methoxybenzophenone, 2-phenylbenzimidazol-5-sulfonic acid and their potassium, sodium and triethanolamine salts, 1-(4-tert-butylphenyl)-3-(4-methoxyphenyl)propane-1,3-dione, 4-methoxycinnamic acid 2-ethylhexyl ester and 3-(4′-methylbenzylidene)-D,L-camphor.

UV filters show molar extinction coefficient at the absorption maximum is above 15,000, in particular above 20,000 are preferred.

In addition, it has been found that with structurally similar UV filters, in many cases the water-insoluble compound has the greater effect in comparison with water-soluble compounds which differ from them through one or more additional ionic groups within the scope of the teaching according to the invention. Water-insoluble compounds within the scope of the invention are understood to be UV filters which do not dissolve in water at 20° C. in an amount of more than 1 wt %, in particular no more than 0.1 wt %. In addition, these compounds should be at least 0.1 wt % soluble, in particular at least 1 wt % soluble in the usual cosmetic oil components at room temperature. The use of water-insoluble UV filters may therefore be preferred according to the invention.

According to another embodiment of the invention, UV filters having a cationic group, in particular a quaternary ammonium group are preferred.

These UV filters have the general structure U-Q.

The structural part U stands for a group absorbing UV rays. This group may in principle be derived from the known UV filters mentioned above which can be used in the cosmetic field in that a group, usually a hydrogen atom, of the UV filter is replaced by a cationic group Q, in particular with a quaternary amino function.

Compounds from which structural part U can be derived include for example:
substituted benzophenones
p-aminobenzoic acid esters
diphenylacrylic acid esters
cinnamic acid esters
salicylic acid esters
benzimidazoles and
o-aminobenzoic acid esters.

Structural parts U which are derived from cinnamic acid amide or from N,N-dimethylaminobenzoic acid amide are preferred according to the invention.

The structural parts U may in principle be selected so that the absorption maximum of the UV filters may lie in the UVA (315 400 nm) range as well as in the UVB (280-315 nm) range or in the UVC (<280 nm) range. UV filters with an absorption maximum in the UVB range, in particular in the range of approx. 280 to approx. 300 nm are especially preferred.

In addition the structural part U, also depending on the structural part Q, is preferably selected so that the molar extinction coefficient of the UV filter at the absorption maximum is above 15,000, in particular above 20,000.

The structural part Q preferably contains as the cationic group a quaternary ammonium group. This quaternary ammonium group may in principle be connected directly to the structural part U, so that the structural part U represents one of the four substituents of the positively charged nitrogen atom.

However one of the four substituents on the positive charged nitrogen atom is preferably a group, in particular an alkylene group with 2 to 6 carbon atoms, which functions as a compound between the structural part U and the positively charged nitrogen atom.

The group Q advantageously has the general structure (CH₂)ₓ—N⁺R¹R²R³X⁻ in which x stands for an integer from 1 to 4, R¹ and R² independently of one another stand for C₁₋₄ alkyl groups, R³ stands for a C₁₋₂₂ alkyl group or a benzyl group and X⁻ stands for a physiologically tolerable anion. Within the context of this general structure, x preferably stands for the number 3, R¹ and R² each stand for a methyl group and R³ stands either for a methyl group or a saturated or unsaturated, linear or branched hydrocarbon chain with 8 to 22 carbon atoms, in particular 10 to 18 carbon atoms.

Physiologically tolerable anions include for example inorganic anions such as halides, in particular chloride, bromide and fluoride, sulfate ions and phosphate ions as well as organic anions such as lactate, citrate, acetate, tartrate, methosulfate and tosylate.

Two preferred UV filters with cationic groups include the compounds cinnamic acid amidopropyltrimethylammonium chloride (Inroquat® UV-283) and dodecyldimethylaminobenzamidopropyldimethylammonium tosylate (Escalol® HP 610) which are available as commercial products.

The teaching according to the invention of course also includes the use of a combination of multiple UV filters. Within the scope of this embodiment, the combination of at least one water-insoluble UV filter with at least one UV filter having a cationic group is preferred.

The UV filters (I) are usually contained in the agents according to the invention in amounts of 0.1-5 wt %, based on the total agent. Amounts of 0.4-2.5 wt % are preferred.

The agents according to the invention may also contain a 2-pyrrolidinone-5-carboxylic acid and its derivatives (J). The sodium, potassium, calcium, magnesium or ammonium salts in which the ammonium ion also has one to three C₁ to C₄ alkyl groups in addition to hydrogen are preferred. The sodium salt is most especially preferably preferred. The quantities used in the agents according to the invention preferably amount to 0.05 to 10 wt %, based on the total agent, especially preferably 0.1 to 5 and in particular 0.1 to 3 wt %.

Finally, the agents according to the invention may also contain plant extracts (L).

These extracts are usually prepared by extraction of the whole plant. However in individual cases it may also be preferable to prepare the extracts exclusively from flowers and/or leaves of the plants.

With regard to the plant extracts that can be used according to the invention, reference is made in particular to the extracts which are listed in the table beginning on page 44 of the 3^{rd} edition of the Guidelines for Declaration of Ingredients of Cosmetic Agents, published by the Industrial Association for Body Care and Washing Agents, Inc. (IKW), Frankfurt.

According to the invention, in particular the extracts from green tea, oak bark, stinging nettle, witch hazel, hops, henna, chamomile, burdock root, horsetail, hawthorne, lime-tree blossoms, almond, aloe vera, pine needle, horse chestnut, sandalwood, juniper berry, coconut, mango, apricot, lime, wheat, kiwi, melon, orange, grapefruit, sage, rosemary, birch, mallow, lady's smock, wild thyme, yarrow, thyme, melissa, rest harrow, coltsfoot, marsh mallow, meristem, ginseng and ginger root are preferred.

Especially preferred are the extracts of green tea, oak bark, stinging nettle, witch hazel, hops, chamomile, burdock root, horsetail, lime-tree blossoms, almond, aloe vera, coconut, mango, apricot, lime, wheat, kiwi, melon, orange, grapefruit, sage, rosemary, birch, mallow, lady's smock, wild thyme, yarrow, rest harrow, meristem, ginseng and ginger root.

Most especially suitable for use according to the invention are the extracts of green tea, almond, aloe vera, coconut, mango, apricot, lime, wheat, kiwi and melon.

The plant extracts may be used in both pure form and dilute form according to the invention. If they are used in dilute form they usually contain approx. 2-80 wt % active substance and as the solvent they contain the extraction agent or extraction agent mixture used to obtain them.

It may also be preferable to use mixtures of several different plant extracts in particular two different plant extracts in the agents according to the invention.

In addition, it may prove advantageous if the agents according to the invention contain penetration aids and/or swelling agents (M). These include in particular urea and urea derivatives, guanidine and its derivatives, arginine and its derivatives, water glass, imidazole and its derivatives, histidine and its derivatives, benzyl alcohol, glycerol, glycol and glycol ether, propylene glycol and propylene glycol ether for example propylene glycol monoethyl ether, carbonates, bicarbonates, diols and triols and in particular 1,2-diols and 1,3-diols, for example 1,2 propanediol, 1,2-pentanediol, 1,2-hexanediol, 1,2-dodecanediol, 1,3 propanediol, 1,6-hexanediol, 1,5-pentanediol, 1,4-butanediol.

In the sense of this invention short-chain carboxylic acids (N) may also advantageously support the active ingredient complex (A). Short-chain carboxylic acids and their derivatives in the sense of the invention are understood to be carboxylic acids which may be saturated or unsaturated and/or linear or branched or cyclic and/or aromatic and/or heterocyclic and have a molecular weight of less than 750. In the sense of the invention both saturated or unsaturated, linear or branched carboxylic acids with a chain length of 1 to 16 carbon atoms in the chain are preferred, and those with a chain length of 1 to 12 carbon atoms in the chain are most especially preferred.

The short-chain carboxylic acids in the sense of the invention may have one, two, three or more carboxyl groups. Carboxylic acids with several carboxyl groups in particular di- and tricarboxylic acids are preferred in the sense of the invention. The carboxyl groups may be entirely or partially present as esters, ester anhydride, lactone, amide, imidic acid, lactam, lactim, dicarboximide, carbohydrazide, hydrazone, hydroxame, hydroxime, amidine, amidoxime, nitrile, phosphonate esters or phosphate esters. The carboxylic acids used according to the invention may of course also be substituted along the carbon chain or the ring structure. The substituents of the carboxylic acids that may be used according to the invention include for example C₁-C₈ alkyl, C₂-C₈ alkenyl, aryl, aralkyl and aralkenyl, hydroxymethyl, C₂-C₈ hydroxyalkyl, C₂-C₈ hydroxyalkenyl, aminomethyl, C₂-C₈ aminoalkyl, cyano, formyl, oxo, thioxo, hydroxyl, mercapto, amino, carboxyl or imino groups. Preferred substituents include C₁-C₈ alkyl, hydroxymethyl, hydroxyl, amino and carboxyl groups. Substituents in the α-position are especially preferred. Most especially preferred substituents include hydroxyl, alkoxy and amino groups, where the amino function may optionally also be substituted by alkyl, aryl, aralkyl and/or alkenyl radicals. Also preferred carboxylic acid derivatives include phosphonate and phosphate esters.

The agents according to the invention have advantageous properties and also impart advantageous properties to the hair treated with them. Advantages have been observed in particular in conditioning hair. Hair treatment agents according to the invention thus improve the feel and combability of both wet and dry hair treated with them. This can also be observed to prevent early formation of split ends in the treated air without any negative effect on the volume or body.

Another subject matter of the present invention is therefore a method for care of human air in which an agent according to the invention is applied to the hair, left on the hair for a treatment time of 10 to 600 seconds, preferably 30 to 150 seconds, and then rinsed out of the hair.

In addition, the subject matter of the invention is a method for caring for human hair in which an agent according to the invention is applied to the hair and remains there until the next shampooing, i.e., it is not rinsed out again after a treatment time of a few seconds.

This procedure according to the invention is based on a product according to the invention, which is formulated as a so-called “rinse-off product,” i.e., the product remains in the hair for a period of time of less than a quarter hour and is then rinsed out. Care results that meet or even exceed the results achieved with traditional leave-in products are surprisingly achieved with the rinse-off products according to the invention.

With regard to additional preferred embodiments of the methods according to the invention, what was said about the agents according to the invention also applies here, mutatis mutandis.

Another subject matter of the present invention is the use of agents according to the invention for unencumbering hair care.

With regard to additional preferred embodiments of the use according to the invention, what was said about the agents according to the invention also applies here, mutatis mutandis.

### EXAMPLES

Care agents having the following composition (amounts given in wt %, based on the total agent) were prepared:

| | A | B |
|---|---|---|
| Cetrimonium chloride | 0.5 | 0.5 |
| PEG-10 dimethicone | 0.35 | 0.35 |
| Cyclopentasiloxane | 7.0 | 7.0 |
| Dimethiconol | 4.5 | 4.5 |
| Polyquatemium-16 | 1.0 | 1.0 |
| Sodium benzoate | 0.1 | 0.1 |
| D-Panthenol 75% | 0.5 | 0.5 |
| Dow Corning ® 556 | 1.5 | 1.5 |
| Lactic acid 80% | 0.7 | 0.7 |
| Cationic wheat hydrolysate | 1.0 | — |
| Water, deionized | to a total of 100 | to a total of 100 |

The agents were applied to untreated human hair and rinsed out with warm water after 5 minutes. The treated and untreated hair samples were tested with the help of differential thermal analysis, wherein the peak temperature indicates to what extent the matrix and the α-helix are stable. The higher the peak temperature, the more stable is the hair structure.

The hair treated with the agents according to the invention had a higher peak temperature by an average of 3° C. after the treatment (153° C. instead of 150° C.). Hair treated with a conventional leave-in treatment was used for comparison. This comparative sample yielded an increase in peak temperature by only 1° C.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment product, containing, based on its weight:
a) at least 70 wt % water,
b) 0.1 to 1.5 wt % of at least one silicone-based water-in-oil emulsifier from the group of polyethylene glycol-modified silicones, and
c) 1 to 10 wt % cyclic siloxane.

2. The product according to claim 1, comprising—based on its weight—0.15 to 1.25 wt % of the at least one silicone-based water-in-oil emulsifier, which is selected from the group consisting of polyethylene glycol-modified silicones.

3. The product according to claim 1, comprising—based on its weight—1.5 to 9.5 wt % of the cyclic siloxane.

4. The product according to claim 1, wherein the silicone-based water-in-oil emulsifier from the group of polyethylene glycol-modified silicones comprises at least one compound with the INCI designation PEG-x dimethicone, wherein x=2-20.

5. The product according to claim 1, further comprising exclusively cationic compounds as silicone-free emulsifiers.

6. The product according to claim 1, further comprising—based on its weight—0.05 to 7.5 wt % cationic polymer(s), which is at least one polymer selected from the group consisting of:
poly(methacryloyloxyethyl trimethylammonium chloride) (INCI: polyquaternium-37) and/or
quaternized cellulose derivatives (INCI: polyquaternium 10) and/or
cationic alkyl polyglycosides and/or
cationized honey and/or
cationic guar derivatives and/or
polymeric dimethyldiallylammonium salts and their copolymers with esters and amides of acrylic acid and methacrylic acid and/or
copolymers of vinylpyrrolidone with quaternized derivatives of dialkylaminoalkyl acrylate and methacrylate and/or
vinylpyrrolidone/vinylimidazolium methochloride copolymers and/or
quaternized polyvinyl alcohol and/or
polyquaternium 2 and/or
polyquaternium-7 and/or
polyquaternium-16 and/or
polyquaternium 17 and/or
polyquaternium 18 and/or
polyquaternium 24 and/or
polyquaternium 27.

7. A method for treating human hair, comprising:
applying the product according to claims 1 to the hair, and leaving the product on the hair for a treatment time of 10 to 600 seconds before rinsing the product out from the hair.
